(19) United States
(12) Patent Application Publication
(10) Pub. No.: US 2023/0248220 A1
(43) Pub. Date: Aug. 10, 2023

(54) STERILE UNIT AND MANIPULATOR FOR ROBOTIC SURGERY

(71) Applicant: avateramedical GmbH, Jena (DE)

(72) Inventors: Lothar MITZLAFF, Lagos (PT); Anne-Ruth MEYER, Ilmenau (DE)

(21) Appl. No.: 18/165,170

(22) Filed: Feb. 6, 2023

(30) Foreign Application Priority Data
Feb. 7, 2022 (DE) 10 2022 102 806.2

(51) Int. Cl. A61B 1/00; A61B 34/30; A61B 34/00

(52) U.S. Cl. CPC A61B 1/00142; A61B 34/30; A61B 34/70

(57) ABSTRACT

A sterile unit for the sterile connection of an instrument drive unit to a surgical instrument, including: a base with a circular passageway orifice having a transmission element supported to be rotatable around a rotational axis and axially movable in a direction of the rotational axis in a movement range between first and second end positions. The sterile unit also includes a first fastening mechanism connecting the sterile unit to the instrument drive unit and a second fastening mechanism connecting the sterile unit to the instrument. To improve the coupling between the sterile and instrument drive units, the transmission element has a conical region at a circumferential surface and the passageway orifice has a complementary conical region at a passageway wall. The conical regions are aligned coaxially and contact one another such that a rotation of the transmission element around the rotational axis relative to the base is blocked.

### PRIORITY CLAIM

The present application claims priority to German Patent Application No. 10 2022 102 806.2, filed on Feb. 7, 2022, which said application is incorporated by reference in its entirety herein.

### FIELD OF THE INVENTION

The invention is directed to a sterile unit for the sterile connection of an instrument drive unit to a surgical instrument which comprises a base with at least one circular passageway orifice, in which passageway orifice a transmission element is arranged, the transmission element being supported so as to be rotatable around a rotational axis and axially movable in direction of the rotational axis in a predetermined movement range between a first end position and a second end position. The sterile unit further comprises a first fastening mechanism by which the sterile unit is connected to the instrument drive unit and a second fastening mechanism by which the sterile unit is connected to the instrument. By means of the transmission elements, a torque is transmitted from the instrument drive unit to the surgical instrument. The sterile unit is formed as part of a sterile barrier and accordingly shields the operating area in which the surgical instrument operates from the non-sterile instrument drive unit.

The invention is further directed to a manipulator for robotic surgery. The manipulator comprises an instrument drive unit and a sterile unit for coupling with a surgical instrument. The sterile unit has a base with at least one transmission element which is arranged in a circular passageway orifice associated with the at least one transmission element and supported so as to be rotatable around a rotational axis and axially movable in direction of the rotational axis in a defined movement range between a first end position and a second end position. The manipulator further comprises a first fastening mechanism by which the sterile unit is connected to the instrument drive unit. The instrument drive unit has one or more drives, and a drive is associated with the at least one transmission element. The drive further has an engagement element, and the at least one transmission element has an engagement structure complementing the engagement element. The engagement element can comprise one or more pins, for example, in which case the engagement structure comprises a slot into which one or more pins fit. Other suitable structures as used in the art are also contemplated for this purpose. The transmission element is located in the second end position when the transmission element and the engagement element are aligned with one another rotationally around the rotational axis such that the engagement element engages in the engagement structure and transmits to the engagement structure a torque acting on the engagement element perpendicular to the rotational axis. The transmission element is located in the first end position when the engagement element and the engagement structure are not aligned with one another. In this case, the transmission element lies on the engagement element at the underside and is accordingly raised axially relative to the rotational axis.

### BACKGROUND OF THE INVENTION

Telemanipulators have been successfully used for many decades in diverse environments which cannot be easily accessed by a person. This applies to applications in outer space, underwater applications, nuclear reactors and especially also surgical applications.

The discipline of laparoscopic robotic surgery which combines concepts from robotics with those of virtual reality has become established over the past two decades allowing a physician to carry out minimally invasive procedures on patients using eye-hand coordination.

Instruments used in manual laparoscopic surgery have a long shaft at the distal end of which is located an end effector, e.g., a gripper or scissors, with a handle being located at its proximal end. This handle has a lever, button or similar mechanism by means of which the end effector can be moved. Such instruments are difficult to handle because their range of motion is sharply restricted by the fact that they are guided through a small incision in the patient. Also, the physician has difficulty self-orienting because of the need to compensate for a generally deviating viewing angle of the endoscope.

This difficulty can be solved by a telemanipulator. Such a telemanipulator comprises a plurality of robot arms, the manipulators and, generally, two input devices, one for each hand. One of the manipulators holds and controls an endoscope. The other manipulators in each instance hold and control a surgical instrument. By means of suitable transformations, a computer connected to the manipulators and input devices creates the impression for the surgeon that the surgeon is viewing the surgical site from the direction of the endoscope and that the end effectors of the instruments are the surgeon's hands. In this way, the surgeon can make use of eye-hand coordination and operate efficiently.

Whereas the input devices of a telemanipulator are generally arranged away from the patient in the operating room, the manipulators must be arranged close to the patient. However, it is mandatory that this area be kept sterile in order to prevent germs from contaminating the patient's wounds as far as possible. To this end, the manipulators are wrapped with sterile plastic sheeting. However, this is not possible for the instruments which must then usually be directly sterilized. In this case, instruments are used which can be re-sterilized after use or which are disposed of after a single use.

Therefore, it is common practice in the art to outfit manipulators of surgical robotic systems with an instrument drive unit which is wrapped with sterile sheeting. To enable operation of the instruments with this, sterile adapters are used which transmit a force from the drives of the instrument drive unit to the outputs of the instruments by means of transmission elements which are formed as rotating disks. These components are generally coupled with one another by positive engagement, which in turn requires that the transmission elements are aligned with the drives and, finally, with the outputs of the instrument.

A solution to this problem is known from EP 3 119 328 B1 which discloses a sterile adapter having a plurality of rotatably supported transmission elements. In order to connect to the drives of the instrument drive unit, the transmission elements have projections which are formed to engage in the drives. Further, the transmission elements comprise a cutout in each instance which engages with a locking mechanism or lock that fixes the transmission elements in their alignment. By coupling the sterile adapter with the instrument drive unit, the transmission elements are raised so that they either contact the locking mechanism from below or the locking elements immediately engage in the cutouts. In both cases, the drives subsequently rotate. In the first case, the transmission elements are carried along by friction until they can engage in the locking mechanism and lock. The drives rotate until the transmission element is aligned with the drive, i.e., until the projections and the respective cutouts are located one above the other and the transmission element enters into a positive engagement with the respective drive so that a torque is transmitted.

JP 2020-162637 A2 discloses a sterile unit for a surgical system. The sterile unit can be arranged between an instrument drive unit and a surgical instrument. Transmission structures through which a force for actuating, i.e., for robot-supported deflection of, an end effector of the instrument is transmitted to the sterile unit by means of transmission elements are located on sides of the instrument as well as on sides of the instrument drive unit. In this solution, every drive of the instrument drive unit and a transmission element associated with it must be aligned relative to one another. A difference between a friction torque acting on a first contact surface and a friction torque acting on a second contact surface is made use of for this purpose. The first contact surface is on the side of the transmission element remote of the instrument drive unit and contacts a base of the sterile unit at a circumferential edge. The second contact surface is arranged on the side facing the instrument drive unit and, insofar as the transmission element and the associated drive are not aligned relative to one another, contacts an engagement structure at the drive. A friction torque acting on the first contact surface is greater than the friction torque acting on the second contact surface. In this way, a rotation of the transmission element is blocked and the drive can be aligned with the transmission element. After alignment, the engagement element engages in the engagement structure and the transmission element lowers so that the first contact surface no longer contacts the base of the sterile unit. A disadvantage to this solution consists in that the transmission element may become jammed in the base in a construction of this type because of the circumferential edge on the sterile unit and be damaged upon further operation if the sterile unit is not placed evenly on the instrument drive unit. Further, depending on the combination of materials and the pressing pressure acting on the contact surfaces, it can happen that the transmission element nevertheless rotates slowly around its own axis so that the alignment process described above takes longer.

### SUMMARY OF THE INVENTION

Therefore, it is an object of the invention to provide a sterile unit and a manipulator for a surgical robotic system which allows the transmission elements to be connected more easily and facilitates the alignment of the transmission elements and drives.

The above-stated object is met in the sterile unit described in the introduction in that the transmission element has a conical region at a circumferential surface and the passageway orifice has a complementary conical region at a passageway wall, which conical regions are aligned coaxial to one another and contact one another in a positively engaging and frictionally engaging manner in the first end position such that a rotation of the transmission element around the rotational axis relative to the base is blocked. The conical regions act in a self-locking manner such that an increased friction torque is generated. The friction torque is dependent on a normal force which presses the two bodies together and acts perpendicularly on the surface thereof and a friction coefficient between the bodies as material constant. When the transmission element is inserted, the conical shape results in a circumferential elastic deformation of the transmission element and of the base at the conical region because the force bringing them together acts at an angle to the surface. The force opposing this deformation is responsible for the self-locking because it increases the acting normal force and, therefore, the friction torque. Further, the conical regions act as a guide of the transmission element which prevents the transmission element from jamming. At the same time, the transmission elements can be produced in one piece so that the sterile adapter can be composed of few component parts so that production costs can be reduced.

In an advantageous embodiment, the conical regions taper in such a way that the conical regions each form a frustum of a cone, the cone having an apex angle of between 10° and 60°. In this way, an acting normal force is increased and the self-locking is accordingly optimized.

It is further advantageous to roughen the conical region of the transmission element and/or the conical region of the passageway orifice or to provide it with a material which increases the friction coefficient so as to improve the blocking of the transmission element. There are primarily two effects operative in friction between solid bodies: roughness which causes instances of microscopic positive engagement when the solid bodies slide against one another, and adhesion, i.e., frictional engagement due to molecular forces of attraction. Both effects can be influenced by corresponding selection of materials, machining the surfaces or coating the surfaces with suitable materials. An exceptional instance consists in the rubberization of the surface or the introduction of a rubber ring because, with rubber, the friction is highly dependent on the internal friction of an elastomer from which the rubber is made. This is due to the fact that when there is contact between an elastomer and a rigid surface, the friction energy is dissipated by a deformation of the elastomer.

In a further advantageous embodiment, the transmission element is held in the passageway orifice of the sterile unit by means of at least two snap hooks. This further facilitates assembly and reduces production costs. Further, the movement range axial to the rotational axis is realized in a particularly simple manner in that a spacing is achieved between the snap hooks and a projection opposite the snap hooks.

The above-stated object is further met by the manipulator described in the introduction in that the transmission element has a conical region at a circumferential surface and the passageway orifice has a complementary conical region at a passageway wall, which conical regions are aligned coaxial to one another and contact one another in a positively engaging and frictionally engaging manner in the first end position such that a rotation of the transmission element around the rotational axis relative to the base is blocked. When the sterile unit is connected to the instrument drive unit, the engagement element must engage in the engagement structure before the instrument can be controlled. As soon as the sterile unit is fastened by means of the fastening element to the instrument drive unit, the drives rotate the engagement element around the rotational axis. Since the transmission element is located in the second end position in this case and a rotation relative to the base is blocked by positive engagement and frictional engagement of the conical regions, the engagement element slides along the underside of the transmission element. As soon as the engagement element and the engagement structure are aligned relative to one another, the transmission element drops onto the engagement element resulting in an engagement and, as a result, a torque generated at the drive acts on the transmission element and is subsequently transmitted through the latter to the instrument.

In an advantageous embodiment, the conical regions taper in such a way that the conical regions form a frustum of a cone in each instance, the cone having an apex angle of between 10° and 60°. In this way, the acting normal force is increased and the self-locking is accordingly optimized.

In a further advantageous embodiment, the friction coefficient between the conical region of the transmission element and the conical region of the passageway orifice is greater than the friction coefficient between an underside of the transmission element and the engagement element. In the second end position, the conical region of the transmission element is pressed on the conical region of the passageway orifice. In order to block the rotation of the transmission element around its own axis in this way, the friction coefficients must differ to a sufficient extent that the transmission element either does not move at all while the drive orients the engagement element or the transmission element at least rotates more slowly than the drive.

In order to adapt the friction coefficients, it is particularly advantageous when the conical region of the transmission element and/or the conical region of the passageway orifice is roughened or provided with a material which increases the friction coefficient. As was already detailed above, the transmission element is also blocked to a greater degree in this advantageous construction.

It is also advantageous for this inventive solution if the transmission element is held in the passageway orifice by at least two snap hooks because this facilitates assembly and can lower production costs.

It should be understood that the features mentioned above and those yet to be described hereinafter can be used not only in the stated combinations, but also in other combinations or alone, without departing from the scope of the present invention.

### BRIEF DESCRIPTION OF THE DRAWINGS

The invention will be described in more detail in the following based on exemplary embodiments with reference to the accompanying drawings which likewise disclose features key to the invention. These embodiment examples are to be considered merely as illustrative and not restrictive. For example, it is not to be construed from a description of an embodiment example having a plurality of elements or components that all of these elements or components are necessary to its implementation. On the contrary, other embodiment examples can also contain alternative elements and components, fewer elements or components or additional elements or components. Elements or components of different embodiment examples can be combined unless stated to the contrary. Modifications and variations which are described for one of the embodiment examples may also be applicable to other embodiment examples. In order to avoid repetition, like or comparable elements are designated by like reference numerals in different figures and are not described repeatedly. The drawings show:

FIG. 1 a manipulator with an instrument drive unit, a sterile unit in a first embodiment, and a surgical instrument;

FIG. 2 the instrument drive unit, the sterile unit in a first embodiment and the surgical instrument in an exploded view;

FIG. 3A a second embodiment of the sterile unit in a first view;

FIG. 3B a second embodiment of the sterile unit in a second view;

FIG. 3C a detailed view of a transmission element;

FIG. 4 a sectional view of the second embodiment of the sterile unit.

### DETAILED DESCRIPTION

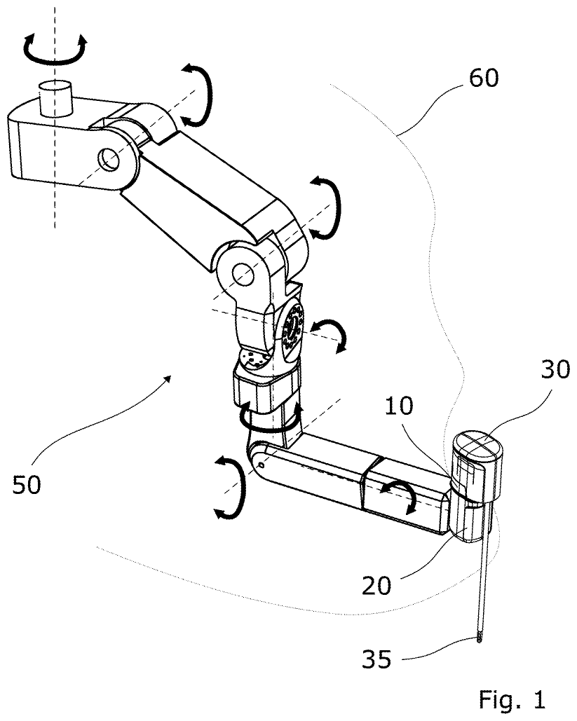

FIG. 1 shows a robotic manipulator 50 with an instrument drive unit 20 and an instrument 30. Such manipulators 50 comprise a plurality of joints which allow the instrument 30 to be freely positioned in space. In this regard, they can be employed independently as aids for the surgeon, e.g., as endoscope holders, or in combination with a surgical telemanipulator. Since the area surrounding a patient to be operated on must always be sterile so as to prevent infections, parts of the system which are difficult to sterilize, i.e., the manipulator 50 and the instrument drive unit 20, are separated from the sterile zone by a sterile barrier 60. As described above, the sterile unit 10 shown here in a first embodiment is formed as an adaptor so that an end effector 35 of the instrument 30 can nevertheless by actuated.

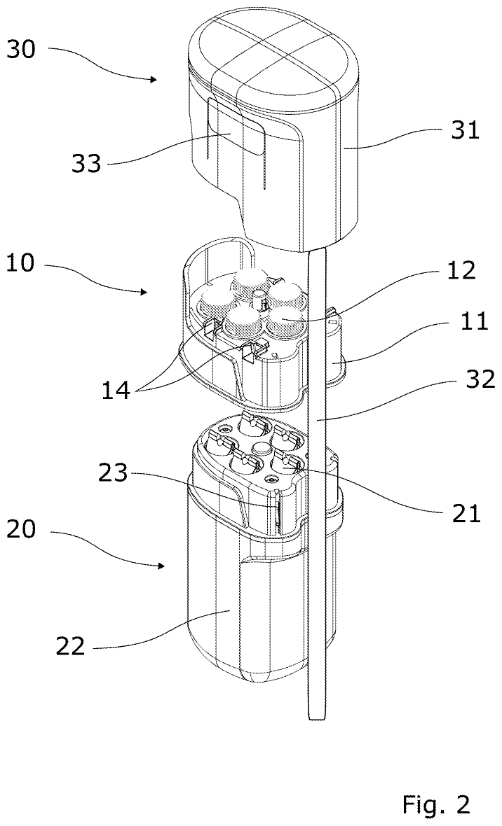

FIG. 2 shows how the instrument 30, the first embodiment of the sterile unit 10 and the instrument drive unit 20 are arranged relative to one another. The sterile unit 10 has a base 11 and a plurality of transmission elements 12 which are supported so as to be rotatable around a rotational axis. The sterile unit 10 is arranged between the instrument 30 and the instrument drive unit 20.

The surgical instrument 30 comprises an instrument housing 31 and an instrument shaft 32. In this instance, there is no end effector 35 arranged at a distal end of the instrument shaft 32 because this is an endoscope. Generally, however, all types of instruments 30, for example, grippers, scissors, staplers, or dissectors, can be used with the corresponding end effectors 35. The quantity of degrees of freedom depends on the type of instrument 30. Apart from the movement of end effector joints, a mechanical triggering of additional functions, for example, the activation of a blade, is also included. As a rule, the quantity of degrees of freedom is between one, for example, for an endoscope, or five degrees of freedom for a stapler. However, the quantity of degrees of freedom is optional in principle and can be adapted to the specific case of application.

The instrument drive unit 20 comprises a drive housing 22 in which are accommodated five drives 21, each of which has an engagement element and a motor, not shown here, generally an electric motor which generates a torque. The engagement element is arranged outside of the drive housing 22. A more exact description follows below. The torque is transmitted by the respective engagement element to the transmission elements 12 which have an engagement structure complementing the engagement element. On the side facing the instrument 30, the transmission elements 12 are provided with a selected coupling structure by which the torque is conveyed to outputs of the instrument 30, not visible here, in order to move the end effector 35. In the present example, the coupling structure is formed as a knurled circumferential surface of the respective transmission element 12. However, other variants are also used in the art, all of which can also be applied here in place of the coupling structure.

In addition to the drives 21 and the drive housing 22, the instrument drive unit 20 comprises one or more first fastening elements 23 which fasten the sterile unit 10 to the instrument drive unit 20. For this purpose, a complementary first fastening mechanism or fastener, not visible in this drawing, is in turn provided at the sterile unit 10. Located on the side of the sterile unit 10 facing the instrument 30 is a second fastening mechanism 14 which in turn allows the surgical instrument 30 to be fastened to the sterile unit 10. This connection can be undone by means of a release mechanism 33.

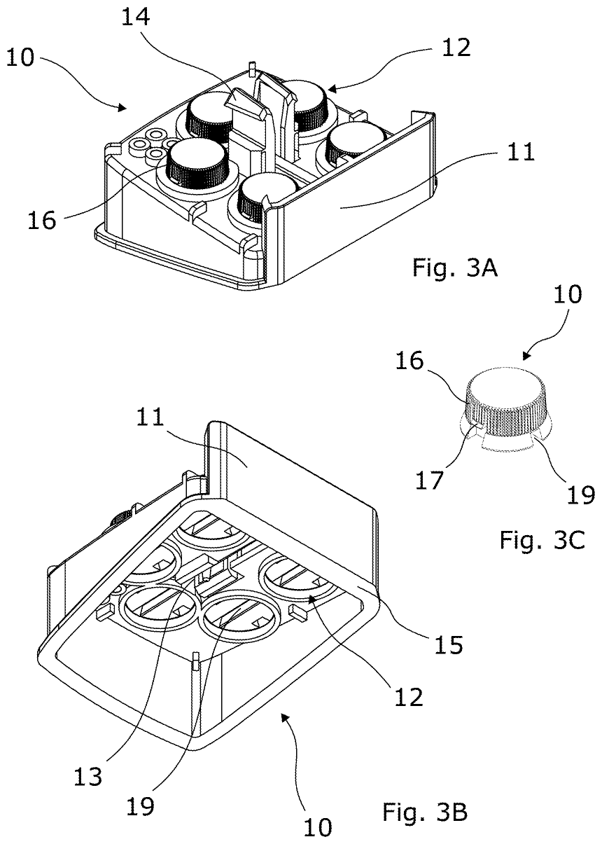

FIGS. 3A and 3B show the sterile unit 10 in more detail in a second embodiment. Apart from the transmission elements 12 and the base 11, a first fastening mechanism 13 and a second fastening mechanism 14 are arranged on the sterile unit 10. The first fastening mechanism 13 which allows the sterile unit 10 to be fastened to the instrument drive unit 20 by means of the first fastening elements 23 located at the latter is disposed on the side of the sterile unit 10 which is associated with the instrument drive unit 20 and shown in FIG. 3B. The second fastening mechanism 14 which allows the surgical instrument 30 to be fastened to the sterile unit 10 via second fastening elements located at the instrument 30 is disposed on the side of the sterile unit 10 which faces the instrument 30 and can be seen in FIG. 3A. Both fastening mechanisms 13, 14 are parts of a snap-in connection, but the first fastening mechanism 13 and second mechanism 14 can generally be formed in any manner customary in the art insofar as the sterile unit 10 can be detachably or permanently connected to the instrument 30 and the instrument drive unit 20 by frictional engagement or positive engagement.

The transmission element 12 is shown in more detail in FIG. 3C and is inserted into a passageway orifice of the base 11. There are various possibilities for the concrete realization thereof. For example, the base 11 can be formed of two parts and the transmission element 12 is received between the two parts of the base 11. Alternatively, as is shown in FIGS. 3A, 3B and 3C, the transmission element 12 itself is outfitted with a fastening option. It can be formed itself of two bodies between which the base 11 is clamped, or it can be outfitted with fastening means which hold it on the base 11 so as to be rotatable around the rotational axis. The snap hooks 17 shown in FIG. 3C, ball joint connections or the like fastening means are suitable for this purpose.

Although not shown, other possibilities include differently shaped transmission elements 12 or an electric contact for conducting current signals through the sterile unit 10. Further, a fastening surface 15 is preferably provided at the side of the base 11 of the sterile unit 10 facing the instrument drive unit 20. In this case, a sterile sheeting is applied by means of glue or welding and, together with the sterile unit 10, forms a sterile barrier 60. Another possibility customary in the art for introducing the sterile sheeting consists in forming the base 11 from two parts so that the sterile sheeting is clamped between the two parts.

The transmission elements 12 have a coupling structure 16 at the upper side thereof which corresponds to the embodiment form in FIG. 2. At the underside which is visible in FIG. 3B, the transmission elements 12 have an engagement structure 19 for receiving an engagement element of the associated drive 21.

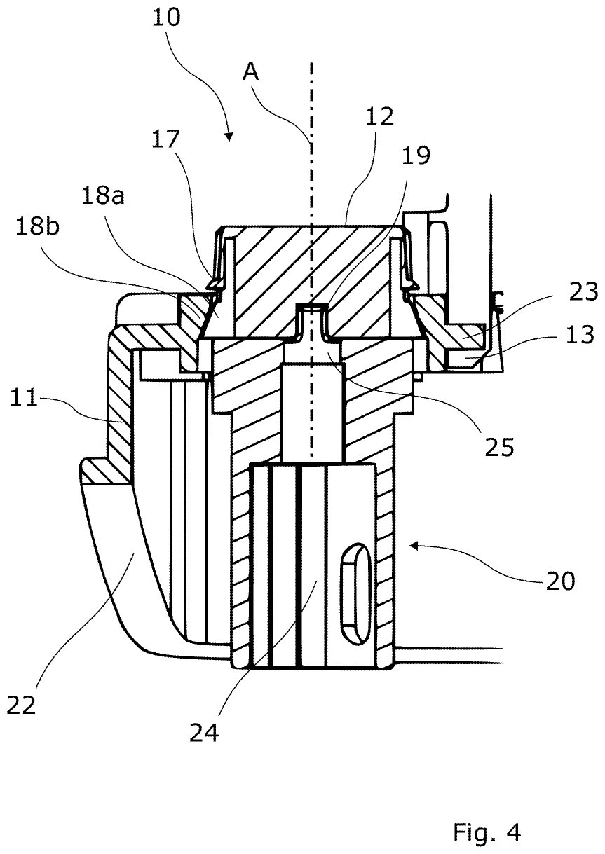

FIG. 4 shows a section through an instrument drive unit 20 connected to the sterile unit 10 at the location of a transmission element 12. The sterile unit 10 is fastened to the first fastening element 23 of the instrument drive unit 20 by means of the first fastening mechanism 13. In the present example, this is a snap-in connection. A sterile sheeting 60, not shown here, which is connected to the sterile unit 10 is arranged between the base 11 and the drive housing 22. The transmission element 12 is mounted in a passageway orifice and is held at the upper side by the snap hook 17. On the opposite side facing the instrument drive unit 20, the transmission element 12 has a first conical region 18a at a circumferential surface. Also located at the base 11 is a second conical region 18b on a passageway wall of the passageway orifice. The two conical regions 18a, 18b are oriented coaxially relative to one another and are complementary so that the transmission element 12 can engage with the base 11 in this location by positive engagement in a planar manner.

The transmission element 12 is movable axially in a predetermined movement range in direction of a rotational axis A between a first end position and a second end position. The end positions depend on the alignment of the engagement structure 19 with respect to an engagement element 25. In this instance, the engagement element 25 is formed as a cube-shaped pin, and the engagement structure 29 is formed as a slot into which the pin fits. Other suitable structures customary in the art are also contemplated. For example, a plurality of pins and complementary slots as well as cross-shaped, star-shaped or polygonal structures are also contemplated. The side walls of the engagement structure 19 and of the engagement element 25 are advantageously oriented parallel to the rotational axis A in order to ensure a good transmission of the torque introduced by the drive 21. An inverse arrangement in which, for example, the engagement structure 19 is formed as a pin and the engagement element 25 is formed as a slot into which the pin fits is also easily possible.

If the sterile unit 10 and the instrument drive unit 20 are connected to one another, the engagement structure 19 and the engagement element 25 are generally not aligned with one another and do not engage in one another. The transmission of a torque and, therefore, the driving of the surgical instrument 30 is accordingly impossible. The transmission element 12 rests by an underside on the engagement element 25 so that it is raised and located in the first end position. The complementary conical regions 18a, 18b are connected by positive engagement and frictional engagement in the first end position.

In order to transmit a torque from a motor 24 of the drive 21 of the instrument drive unit 20 to the transmission element 12, the engagement element 25 must be aligned with the engagement structure 19 so that they engage in one another. This is brought about by the conical regions 18a, 18b which act in a self-locking manner in the first end position by means of the positive engagement and frictional engagement. This effect can be further amplified by different friction coefficients and friction radii between the engagement element 25 and the engagement structure 19. The system is generally so configured that a first friction torque acting between the engagement element 25 and the underside of the transmission element 12 is less than a second friction torque acting between the conical regions 18a, 18b. If the conical regions 18a, 18b are described as a truncated cone, the cone from which the truncated cone is taken has an apex angle. The more acute this apex angle, the larger the first friction torque. If the apex angle falls short of a critical apex angle which is empirically approximately 8° to 10° and also depends on the selected materials, the transmission element 12 cannot disengage from the first end position without being acted upon by an external force, and an alignment is no longer possible. If, on the other hand, the apex angle is larger, the first friction torque decreases until the advantage resulting from the conical regions 18a, 18b empirically and also depending on the materials used when the angle of approximately 60° to 80° is exceeded can be forfeited. In addition, the friction coefficient between the conical region 18a of the transmission element 12 and the conical region 18b of the passageway orifice can be greater than the friction coefficient between the underside of the transmission element 12 and the engagement element 25. This can be achieved in that the conical region 18a of the transmission element 12 and/or the conical region 18b of the passageway orifice are/is roughened or provided with a material that increases the friction coefficient, while the underside of the transmission element 12 and the engagement element 25 are smooth.

If the complementary conical regions 18a, 18b are connected in positive engagement and frictional engagement, then either the transmission element 12 does not rotate around the rotational axis A while the engagement element 25 slides over the underside of the transmission element 12 or, alternatively, the transmission element 12 rotates slower than the engagement element 25. In both of these cases, the rotation of the transmission element 12 around the rotational axis A relative to the base 11 is blocked, and the engagement element 25 is continually adapted with respect to its position relative to the transmission element 12 through a rotation by means of the motor 24 because it rotates faster than the transmission element 12.

When a position is reached in which the transmission element 12 and the engagement element 25 are so oriented to one another rotationally around the rotational axis A that the engagement element 25 engages in the engagement structure 19, the transmission element 12 enters the second end position. The engagement element 25 is received by the engagement structure 19 so that a torque acting on the engagement element 25 perpendicular to the rotational axis A is transmitted to the engagement structure 19 and, accordingly, to the transmission element 12. Since the conical regions 18a, 18b are no longer connected by positive engagement and frictional engagement, friction torque no longer acts at this location, and the transmission element 12 can rotate freely about the rotational axis A and transmit the torque virtually without losses.

### REFERENCE CHARACTERS

10 sterile unit
11 base
12 transmission element
13 first fastening mechanism
14 second fastening mechanism
15 fastening surface
16 coupling structure
17 snap hook
18a, b conical region
19 engagement structure
20 instrument drive unit
21 drive
22 drive housing
23 first fastening element
24 motor
25 engagement element
30 surgical instrument
31 instrument housing
32 instrument shaft
33 release mechanism
35 end effector
50 manipulator
60 sterile barrier
A rotational axis

What is claimed is:

1. A sterile unit for the sterile connection of an instrument drive unit to a surgical instrument, comprising:
a base with at least one circular passageway orifice, wherein a transmission element is arranged in the passageway orifice, which transmission element is supported so as to be rotatable around a rotational axis and axially movable in a direction of the rotational axis in a predetermined movement range between a first end position and a second end position, and
a first fastening mechanism configured to connect the sterile unit to the instrument drive unit and a second fastening mechanism configured to connect the sterile unit to the instrument,
wherein the transmission element has a conical region at a circumferential surface and the passageway orifice has a complementary conical region at a passageway wall, which conical regions are aligned coaxially relative to one another and contact one another in a positively-engaging and frictionally-engaging manner in the first end position such that a rotation of the transmission element around the rotational axis relative to the base is blocked.

2. The sterile unit according to claim 1, wherein the conical regions each form a frustum of a cone, wherein the cone has an apex angle of between 10° and 60°.

3. The sterile unit according to claim 1, wherein the conical region of the transmission element and/or the conical region of the passageway orifice are/is roughened or provided with a material which increases the friction coefficient.

4. The sterile unit according to claim 2, wherein the conical region of the transmission element and/or the conical region of the passageway orifice are/is roughened or provided with a material which increases the friction coefficient.

5. The sterile unit according to claim 1, wherein the transmission element is held in the passageway orifice by means of at least two snap hooks.

6. The sterile unit according to claim 2, wherein the transmission element is held in the passageway orifice by means of at least two snap hooks.

7. The sterile unit according to claim 3, wherein the transmission element is held in the passageway orifice by means of at least two snap hooks.

8. A manipulator for robotic surgery, comprising:
an instrument drive unit, and
a sterile unit for coupling with a surgical instrument, the sterile unit including a base with at least one transmission element which is arranged in a circular passageway orifice associated with the at least one transmission element and which is supported so as to be rotatable around a rotational axis and axially movable in a direction of the rotational axis in a defined movement range between a first end position and a second end position, and a first fastening mechanism by which the sterile unit is connected to the instrument drive unit, and
wherein the instrument drive unit has one or more drives, a drive being associated with the at least one transmission element,
wherein the drive has an engagement element, and the at least one transmission element has an engagement structure complementing the engagement element, and the transmission element is located in the second end position when the transmission element and the engagement element are aligned with one another rotationally around the rotational axis such that the engagement element engages in the engagement structure and transmits to the engagement structure a torque acting on the engagement element perpendicular to the rotational axis, and the transmission element is located in the first end position when the engagement element and the engagement structure are not aligned with one another, and
wherein the transmission element has a conical region at a circumferential surface and the passageway orifice has a complementary conical region at a passageway wall, and
wherein the conical regions come in contact in a positively engaging and frictionally engaging manner in the first end position such that a rotation of the transmission element around the rotational axis relative to the base is blocked.

9. The manipulator according to claim 8, wherein the conical regions each form a frustum of a cone, and wherein the cone has an apex angle of between 10° and 60°.

10. The manipulator according to claim 8, wherein a friction coefficient between the conical region of the transmission element and the conical region of the passageway orifice is greater than a friction coefficient between an underside of the transmission element and the engagement element.

11. The manipulator according to claim 9, wherein a friction coefficient between the conical region of the transmission element and the conical region of the passageway orifice is greater than a friction coefficient between an underside of the transmission element and the engagement element.

12. The manipulator according to claim 10, wherein the conical region of the transmission element and/or the conical region of the passageway orifice are/is roughened or provided with a material which increases the friction coefficient.

13. The manipulator according to claim 8, wherein the transmission element is held in the passageway orifice by at least two snap hooks.

14. The manipulator according to claim 9, wherein the transmission element is held in the passageway orifice by at least two snap hooks.

15. The manipulator according to claim 10, wherein the transmission element is held in the passageway orifice by at least two snap hooks.

16. The manipulator according to claim 11, wherein the transmission element is held in the passageway orifice by at least two snap hooks.

17. The manipulator according to claim 12, wherein the transmission element is held in the passageway orifice by at least two snap hooks.
